(19) United States
(12) Patent Application Publication
(10) Pub. No.: US 2006/0110325 A1
(43) Pub. Date: May 25, 2006

(54) Serotonin and catecholamine segment optimization technology

(76) Inventor: Martin C. Hinz, Duluth, MN (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 11/282,965

(22) Filed: Nov. 18, 2005

Related U.S. Application Data
(63) Continuation-in-part of application No. 10/785,158, filed on Feb. 23, 2004.
(60) Provisional application No. 60/449,229, filed on Feb. 21, 2003.

(51) Int. Cl. A61K 49/00; A61K 31/405

(52) U.S. Cl. 424/9.1; 514/419

(57) ABSTRACT

Methods of using amino acid precursors of the serotonin and catecholamine neurotransmitter systems and laboratory urinary assay of serotonin and catecholamine neurotransmitter levels for optimal treatment of neurotransmitter dysfunction and dysfunction of systems regulated or controlled by the serotonin and/or catecholamine neurotransmitter systems. The methods may also include determining a urinary neurotransmitter phase response to a change in dosing of supplemental amino acid precursors of the serotonin and catecholamine neurotransmitters to optimally treat neurotransmitter dysfunction and dysfunction of systems regulated or controlled by the serotonin and/or catecholamine neurotransmitter systems.

### CROSS-REFERENCE TO RELATED APPLICATIONS

This application is a continuation-in-part of the inventor's co-pending application U.S. patent application Ser. No. 10/785,158, filed Feb. 23, 2004, entitled, “SEROTONIN AND CATECHOLAMINE SYSTEM SEGMENT OPTIMIZATION TECHNOLOGY,” which claims priority to U.S. Provisional Application No. 60/449,229, filed on Feb. 21, 2003, and this application claims priority to U.S. Provisional Application No. 60/715,644, filed on Sep. 9, 2005, all of which are incorporated herein in their entirety by reference.

### 37 C.F.R. § 1.71(e) AUTHORIZATION

A portion of the disclosure of this patent document contains material which is subject to copyright protection. The copyright owner has no objection to the facsimile reproduction by anyone of the patent document or the patent disclosure, as it appears in the U.S. Patent and Trademark Office patent file or records, but otherwise reserves all copyright rights whatsoever.

### FIELD OF THE INVENTION

The present invention relates, generally, to biomedical technology. More particularly, the invention relates to a technology for optimizing the serotonin and catecholamine neurotransmitter systems by administration of amino acid precursors of the serotonin and catecholamine systems in conjunction with serial laboratory assays of the neurotransmitters of the catecholamine and serotonin systems. Most particularly, the invention relates to safe, effective compositions, methods, therapies and techniques for managing the phases of urinary neurotransmitter responses in subjects with serotonin and catecholamine neurotransmitter systems in order to optimize individual and group outcomes in the treatment of neurotransmitter dysfunction and dysfunction of systems regulated or controlled by the serotonin and/or catecholamine systems. The compositions, methods and techniques of the invention have broad applicability with respect to neurotransmitter dysfunction, including disease. The compositions, methods, and techniques may also be useful in other fields.

### BACKGROUND OF THE INVENTION

With administration of amino acid precursors of the serotonin and/or catecholamine system there is an increase in neurotransmitter levels of those systems in the system as a whole. Prior to this work laboratory measurement of neurotransmitters of the catecholamine system (dopamine, norepinephrine, and epinephrine) and the serotonin system (hereafter referred to collectively as “The System”) in correlation with amino acid response for clinical applications had not been calibrated for use. Instead, utilization of amino acid precursors of the serotonin and catecholamine neurotransmitter systems produced random, mixed, and inconsistent results. The present invention provides a methodology for performing meaningful neurotransmitter laboratory assay in support of amino acid therapy in the treatment of neurotransmitter dysfunction of the serotonin and catecholamine neurotransmitter systems. This invention is a preferred step in any process where manipulations of the serotonin and catecholamine neurotransmitter systems are of consideration.

The catecholamine and serotonin neurotransmitters are synthesized in the peripheral and central nervous systems, the kidneys, the liver, the gastrointestinal tract, the lungs, and any other place where the enzymes (i.e., tyrosine hydroxylase, dopa decarboxylase, tryptophan hydroxylase, and 5-hydroxytryptophan hydroxylase) responsible for the conversion of amino acid precursors to neurotransmitters are located. Pathways for serotonin and catecholamine neurotransmitter synthesis are as follows:

Serotonin Synthesis:
L-Tryptophan→L-5-Hydroxytryptophan→Serotonin

Catecholamine Synthesis:
L-Tyrosine→L-Dopa→Dopamine→Norepinephrine→Epinephrine

Neurotransmitter dysfunction associated with the catecholamine and/or serotonin neurotransmitter systems may include, but is not limited to, depression, anxiety, panic attacks, migraine headache, obesity, bulimia, anorexia, premenstrual syndrome, menopause, insomnia, hyperactivity, attention deficit disorder (ADD), impulsivity, obsessionality, inappropriate aggression, inappropriate anger, psychotic illness, obsessive compulsive disorder (OCD), fibromyalgia, addictions, sexual dysfunction, chronic fatigue syndrome, chronic pain states, adrenal fatigue/burnout, attention deficit hyperactivity disorder (ADHD), Parkinsonism, traumatic brain injury, claustrophobia, tension headaches, nocturnal myoclonU.S., irritable bowel syndrome/Chron's disease, states of decreased cognitive function such as dementia and Alzheimer's disease, states associated with aging such as deterioration of organ systems innervated by the serotonin and/or catecholamine systems and deterioration of cognitive functions, hormone dysfunction problems innervated by the serotonin and/or catecholamine systems; cortisol dysfunction related problems, and neurotransmitter reaction to chronic stress.

Applicant has disclosed therapies for treatment of obesity and for serotonin and catecholamine system segment optimization technology in the following U.S. patents and published U.S. patent applications: U.S. Pat. Nos. 6,660,777 issued Dec. 9, 2003; U.S. Pat. No. 6,548,551, issued Apr. 15, 2003; U.S. Pat. No. 6,403,657, issued Jun. 11, 2002; U.S. Pat. No. 6,384,088, issued May 7, 2002; U.S. Pat. No. 6,759,437, issued on Jul. 6, 2004; U.S. Patent Application Publication No. U.S. 2003/0181509 A1, published Sep. 25, 2003; U.S. Patent Application Publication No. U.S. 2002/0094969 A1, published Jul. 18, 2002; U.S. Patent Application Publication No. U.S. 2002/0072537 A1, published Jun. 13, 2002; U.S. Patent Application Publication No. U.S. 2002/0065311 A1, published May 30, 2002; U.S. Patent Application Publication No. U.S. 2002/0040054 A1, published Apr. 4, 2002; U.S. Patent Application Publication No. U.S. 2002/0025972 A1, published Feb. 28, 2002; U.S. Patent Application Publication No. U.S. 2004/0101575 A1, published May 27, 2004; and U.S. Patent Application Publication No. U.S. 2005/0233008 A1, published Oct. 20, 2005.

A need is believed to exist for the present invention. All U.S. patents and patent applications, and all other published documents mentioned anywhere in this application are hereby incorporated by reference in their entirety.

### SUMMARY OF THE INVENTION

The present disclosure describes methods of using amino acid precursors of the serotonin and catecholamine neurotransmitter systems and laboratory urinary assay for optimal treatment of neurotransmitter dysfunction and dysfunction of systems regulated or controlled by the serotonin and/or catecholamine neurotransmitter systems.

In a preferred embodiment, the invention pertains to a method of managing distinct urinary neurotransmitter responses in subjects with a serotonin and a catecholamine neurotransmitter system, the method comprising performing a first laboratory assay on a subject that has consumed a first amino acid dosing of serotonin and catecholamine precursors, performing a second laboratory assay on a subject that has consumed a different second amino acid dosing of serotonin and catecholamine precursors, reviewing the first laboratory assay results, reviewing the second laboratory assay results, and adjusting the amino acid dosing range upon reviewing the first and second laboratory assay results to establish the associated serotonin and catecholamine neurotransmitters in an effective therapeutic range. In some embodiments, the method can further comprise adjusting only the serotonin precursor. In some embodiments, the method can further comprise adjusting only the catecholamine precursor. In some embodiments, the method can further comprise adjusting both the serotonin and catecholamine precursors.

In another embodiment, the invention pertains to a method for managing distinct urinary neurotransmitter responses in subjects with a serotonin and catecholamine neurotransmitter system, the method comprising performing a first urinary laboratory assay on a subject that has consumed a first amino acid dosing of serotonin and catecholamine precursors, performing a second urinary laboratory assay on the subject that has consumed a second amino acid dosing of serotonin and catecholamine precursors, reviewing the first urinary laboratory assay results, reviewing the second urinary laboratory assay results, and adjusting the amino acid dosing range to control the inappropriate excretion of the serotonin and catecholamine neurotransmitters into the urine of the subject.

In another embodiment, the method of adjusting an increase in both the serotonin and catecholamine precursors in a subject occurs when the urinary neurotransmitter levels of serotonin and catecholamine decrease as the dosing of serotonin and catecholamine precursors are increased.

In another embodiment, the method of adjusting an increase in both the serotonin and catecholamine precursors in a subject occurs when the urinary neurotransmitter levels of serotonin and catecholamine remain relatively unchanged as the dosing of serotonin and catecholamine precursors are increased.

In another embodiment, the method of adjusting either an increase or decrease in both the serotonin and catecholamine precursors in a subject to stabilize the serotonin and catecholamine in the therapeutic range occurs when the urinary neurotransmitter levels of serotonin and catecholamine increase as the dosing of serotonin and catecholamine precursors are increased.

In another embodiment, the method of adjusting an increase in catecholamine precursors and a decrease in serotonin precursors in a subject occurs when the urinary neurotransmitter levels of serotonin increases and catecholamine decreases as the dosing of serotonin and catecholamine precursors are increased.

In another embodiment, the method of adjusting an increase in serotonin precursors and decrease in catecholamine precursors in a subject occurs when the urinary neurotransmitter levels of serotonin decreases and catecholamine decreases as the dosing of serotonin and catecholamine precursors are increased.

In another embodiment, the method of adjusting an increase in serotonin precursors and decrease in catecholamine precursors in a subject occurs when the urinary neurotransmitter levels of serotonin remain relatively unchanged and catecholamine increases as the dosing of serotonin and catecholamine precursors are increased.

In another embodiment, the method of adjusting a decrease in serotonin precursors and increase in catecholamine precursors in a subject occurs when the urinary neurotransmitter levels of serotonin increase and catecholamine remains relatively unchanged as the dosing of serotonin and catecholamine precursors are increased.

In a further embodiment, the method of adjusting an increase in the serotonin precursors and a decrease in the catecholamine precursors in a subject occurs when the urinary neurotransmitter levels of serotonin and catecholamine increase as the dosing of serotonin and catecholamine precursors are increased but the serotonin precursors are in a therapeutic range and the catecholamine precursors are above the therapeutic range.

In a further embodiment, the method of adjusting an increase in the catecholamine precursors and a decrease in the serotonin precursors in a subject occurs when the urinary neurotransmitter levels of serotonin and catecholamine increase as the dosing of serotonin and catecholamine precursors are increased but the catecholamine precursors are in a therapeutic range and the serotonin precursors are above the therapeutic range.

In a further embodiment, the urinary serotonin and dopamine levels of a subject can each be established in any one of three phases of urinary response to amino acid precursor dosing.

In a further embodiment, the urinary serotonin and dopamine levels of a subject can each be established in the same phase of urinary response to amino acid precursor dosing.

In still a further embodiment, the urinary serotonin and dopamine levels of a subject can each be established in separate phases of urinary response to amino acid precursor dosing.

In still a further embodiment, amino acid precursor dosing may be adjusted to manipulate each of the urinary serotonin and dopamine neurotransmitter levels of a subject anywhere above, below, or in a therapeutic range in one of any three phases of urinary response to amino acid precursor dosing.

In still a further embodiment, a neurotransmitter assay in support of amino acid therapy is provided which insures that proper levels of neurotransmitters are established and when used properly minimizes the risks of neurotransmitter overload during use.

In still a further embodiment, a method of establishing at least one neurotransmitter status point in a subject comprising the steps of determining a subject's health status with respect to neurotransmitter dysfunction, performing an assay of a body fluid of the subject to determine a neurotransmitter level in the fluid, and defining the assayed neurotransmitter level in the fluid as at least one neurotransmitter status point is provided.

In still yet another embodiment, a method of treating a subject for neurotransmitter dysfunction, comprising the steps of performing a first assay of a body fluid of a subject to determine a baseline neurotransmitter level in the body fluid, administering an amino acid precursor of a neurotransmitter to the subject, administering a second assay of a body fluid of the subject to determine whether the neurotransmitter level in the body fluid is within a predetermined therapeutic range of neurotransmitter levels is provided.

### BRIEF DESCRIPTION OF THE DRAWINGS

FIG. 1 summarizes urinary neurotransmitter level statistical data of subjects diagnosed as suffering from one or more neurotransmitter dysfunction disease by a licensed healthcare provider and the subject not taking any amino acid therapy.

FIG. 2 is a graph showing the three phase relationship between urinary neurotransmitter levels of the serotonin and catecholamine neurotransmitter systems and the daily dosing of amino acid precursors of the serotonin and catecholamine systems of a subject diagnosed with neurotransmitter dysfunction.

FIG. 3 is a graph showing a relationship between urinary serotonin levels versus daily dosing of 5-HTP.

FIG. 4 illustrates a reference range of serotonin 100 to 250 micrograms of serotonin per gram of creatinine.

FIG. 5 is an illustration of the effects of using unopposed precursors of dopamine in treatment.

FIG. 6 is a table summarizing two laboratory assay results of urinary serotonin and catecholamine neurotransmitter levels of subjects not taking amino acid therapy during the first assay but were taking amino acid therapy during the second assay.

FIG. 7 is a table summarizing statistical data and the observed changes in urinary serotonin and dopamine neurotransmitter levels between the first and second assay of FIG. 6.

FIG. 8 is a table summarizing two laboratory assay results of urinary serotonin and dopamine neurotransmitter levels of subjects whose amino acid dosing of the serotonin and dopamine precursors were decreased between the two assays.

FIG. 9 is a table summarizing two laboratory assay results of urinary serotonin and dopamine neurotransmitter levels of subjects whose amino acid dosing of the serotonin precursor decreased and the dopamine precursor increased between the two assays.

FIG. 10 is a table summarizing two laboratory assay results of urinary serotonin and dopamine neurotransmitter levels of subjects whose amino acid dosing of the serotonin precursor increased and the dopamine precursor decreased between the two assays.

FIG. 11 is a table summarizing two laboratory assay results of urinary serotonin and dopamine neurotransmitter levels of subjects whose amino acid dosing of the serotonin and dopamine precursors were increased between the two assays.

FIG. 12 is a table summarizing two laboratory assay results of urinary serotonin and dopamine neurotransmitter levels of subjects whose amino acid dosing of the serotonin precursor was not changed and the dopamine precursor increased between the two assays.

FIG. 13 is a table providing a summary of the statistical data in FIGS. 8-12, particularly the change of urinary neurotransmitter levels between the two assays.

FIG. 14 is a table providing the last amino acid precursor dosing of subjects who have had multiple laboratory assays.

FIG. 15 is a table of laboratory assay results of a subject administered amino acid precursors of the serotonin and catecholamine neurotransmitter systems.

FIG. 16 is a table of laboratory assay results of a subject administered amino acid precursors of the serotonin and catecholamine neurotransmitter systems.

FIG. 17 is a table of laboratory assay results of a subject administered amino acid precursors of the serotonin and catecholamine neurotransmitter systems.

FIG. 18 is a table of laboratory assay results of a subject administered amino acid precursors of the serotonin and catecholamine neurotransmitter systems.

FIG. 19 is a table of laboratory assay results of a subject administered amino acid precursors of the serotonin and catecholamine neurotransmitter systems.

FIG. 20 is a table of laboratory assay results of a subject administered amino acid precursors of the serotonin and catecholamine neurotransmitter systems.

### DETAILED DESCRIPTION OF THE PREFERRED EMBODIMENTS

The embodiments of the invention described are intended to be illustrative and not to be exhaustive or limit the invention to the exact forms disclosed. The embodiments are chosen and described so that a person of ordinary skill in the art will be able to understand the invention and the manner and method of using it.

The teachings of this invention, in general, relate to optimizing individual and group outcomes in the treatment of the neurotransmitters of the serotonin and catecholamine (dopamine, norepinephrine, and epinephrine) systems in the management of dysfunction in human beings as guided by laboratory assay of neurotransmitters. However, the teachings may also be useful in any life form where the catecholamine system and the serotonin system with renal systems are found, such as other animals (i.e., dogs, cats, and horses).

1. General Discussion of the Preferred Embodiment

An understanding of the following basic chemical properties of amino acids is helpful to understand laboratory assay of the response to amino acids by The System.

Amino acids of interest include 3-Hydroxy-L-,t,tyrosine (hereafter referred to as L-dopa) and 5-hydroxytryptophan (hereafter referred to as 5-HTP). L-dopa is an amino acid precursor of the catecholamine system (dopamine, norepinephrine, and epinephrine) and 5-HTP is an amino acid precursor of serotonin. Both share unique chemical properties in the human body and other higher forms of life, including:
1. Both can be absorbed into the system in significant amounts after oral ingestion.
2. Being water soluble both cross the blood brain barrier freely.
3. L-dopa is freely converted to dopamine and 5-HTP is freely converted to serotonin when exposed to the enzyme which catalyzes the synthesis of dopamine or serotonin in conjunction with required cofactors.
4. Neither is subject to biochemical regulation in the synthesis of dopamine and serotonin giving the unique ability to establish theoretical neurotransmitter levels of dopamine and serotonin in unlimited amounts.

Other amino acid precursors include tryptophan of the serotonin system and include but are not limited to tyrosine, N-acetyl-1-tyrosine, and phenylalanine of the catecholamine systems. These amino acids share the same chemical properties as L-dopa and 5-HTP with the exception that they are subject to biochemical feedback regulation meaning that only a limited amount of dopamine and serotonin can be produced by the system with their administration. Production of tryptophan is regulated via the serotonin/tryptophan hydroxylase feedback loop and production of L-dopa from tryosine is regulated via the, norepinephrine/tyrosine hydroxylase feed back loop.

Laboratory assay of neurotransmitters of the serotonin and catecholamine systems can be carried out by assay of serum, saliva, urine, or any other method which accurately reflects the neurotransmitter levels of the system. Based on the following discussion, the preferred method is urinary assay.

With regards to neurotransmitter assay of serum, a major limitation is the collection of a sample. It is a fact that neurotransmitter levels fluctuate greatly from minute-to-minute and the mere act of inducing a needle into a subject causes an instantaneous spike in the neurotransmitter levels of the system which makes it impossible to obtain accurate neurotransmitter level readings that are reflective of levels just prior to insertion of the needle. Methods to compensate for this limitation are cumbersome to the point of not being useful in routine evaluation of subjects. For example, to obtain a true baseline neurotransmitter reading from serum the subject should have a central venous catheter inserted and be allowed to lie quietly in a darkened room for 30 minutes at which point a serum sample can be drawn as long as the subject is not disturbed. A second method of obtaining a serum sample that is meaningful in the assay of serum neurotransmitters levels is to place an indwelling catheter in the subject and allow the subject several days to get used to the catheter at which point serum can be obtained. These two methods are not intended to be an exhaustive discussion on the methodology for obtaining valid serum specimens but are intended to illustrate the considerations that need to be made if serum assay of neurotransmitters in support of amino acid administration is opted for.

Salivary assay may be an option but again saliva is subject to minute-to-minute variability of the system as a whole. A method to compensate for the fluctuations in the system is to obtain several (four to six) saliva samples during an approximate time period of 30 minutes then to average the results of the samples. The drawback of this method is that the final reported assay is a coarse approximation at best and the cost is higher since four to six independent tests need to be run, plus it requires specimen collection over a 30 minute period of time without interruption.

The method opted for as the method of choice in assay of neurotransmitter levels is urinary neurotransmitter testing. This assay is not a completely straight forward assay either and must be preformed with adherence to the following considerations. In reporting urinary assay results consideration must be made to compensate for dilution of the urine (specific gravity variance). Simply assaying the neurotransmitters in a given urine sample will not give results of desired meaning due to variance in specific gravity from sample to sample. One method to compensate for variance in specific gravity is to report the results as a neurotransmitter to creatinine ratio. The preferred method is reporting results as micrograms of neurotransmitter per gram of creatinine in the urine. In utilizing urinary laboratory assay of neurotransmitters the problem of minute-to-minute spikes in the neurotransmitter levels is overcome and the results reported are an average of the neurotransmitters levels in the urine since the bladder was last emptied (generally 2 to 3 hours earlier). Other considerations of urinary neurotransmitter assay include, but are not limited to, the urine should not be collected first thing in the morning unless you are assaying neurotransmitter levels during the night. Contrary to the usual method for collection of urine for neurotransmitter assay where a pathologic diagnosis of pheochromocytoma, a serotonin secreting tumor, and the like is being made, the urine used in assay of neurotransmitters in support of amino acid therapy of The System should be collected late in the day (preferably 5 to 6 hours before bed time) when the neurotransmitter levels are at their lowest. In the case where pathologic diagnosis is being made or in lab testing to assist in establishing neurotransmitter levels in the optimal range throughout the day, or to gauge situations of neurotransmitter overload and toxicity it is desirable to collect urine in the AM when neurotransmitter levels are at their highest so as to demonstrate peak levels. Urinary assay of neurotransmitters in support of amino acid therapy of The System should be collected at or near the low point, 5 or 6 hours before bed time, to insure that a neurotransmitter assay is obtained in an effort to ensure that neurotransmitter levels do not drop below levels needed to keep the system free of disease symptoms (a therapeutic range), although collections at other times of the day may yield meaningful results which are less than optimal.

The term “hyperexcretion” is used for the circumstance where the urinary neurotransmitter assay in a subject not under treatment with amino acids is higher than the upper limit of the reference range reported by the laboratory on assay. It has been observed that 23.7% of human subjects tested late in the day (5 to 6 hours before bed time) are hyperexcreting epinephrine. Hyperexcretion of neurotransmitters also occurs with serotonin, norepinephrine, and dopamine. Very little excretion of the serotonin, dopamine, norepinephrine, and epinephrine neurotransmitters into the urine is systemic neurotransmitters that are being excreted into the urine by the kidneys after being filtered by the glomeruli. Instead, the urinary excretion of these neurotransmitters is the result of synthesis of these neurotransmitters by the kidneys, which is then excreted into the urine.

The kidneys uptake the serotonin and catecholamine neurotransmitters as well as the amino acid precursors of the serotonin and catecholamine neurotransmitters in the proximal convoluted tubules cells of the kidneys via the cat ion uptake ports found on the surface of the proximal convoluted renal tubule cells. The uptake of the serotonin and catecholamine neurotransmitters and the amino acid precursors occurs after they are filtered by the glomeruli of the kidneys from the renal arterial blood flow. Once in the proximal convoluted renal tubule cells, the neurotransmitters are metabolized into metabolites and the amino acids are synthesized into new neurotransmitters as verified by radioisotope tagging studies. Once the proximal convoluted renal tubule cells have synthesized the serotonin, dopamine, norepinephrine, and epinephrine neurotransmitters, these neurotransmitters are either (i) excreted into the final concentrated urine where they are measurable by standard laboratory assay, or (ii) secreted into the system via the renal vein. Some of the norepinephrine excreted into the renal vein and in the adrenal glands is converted to epinephrine.

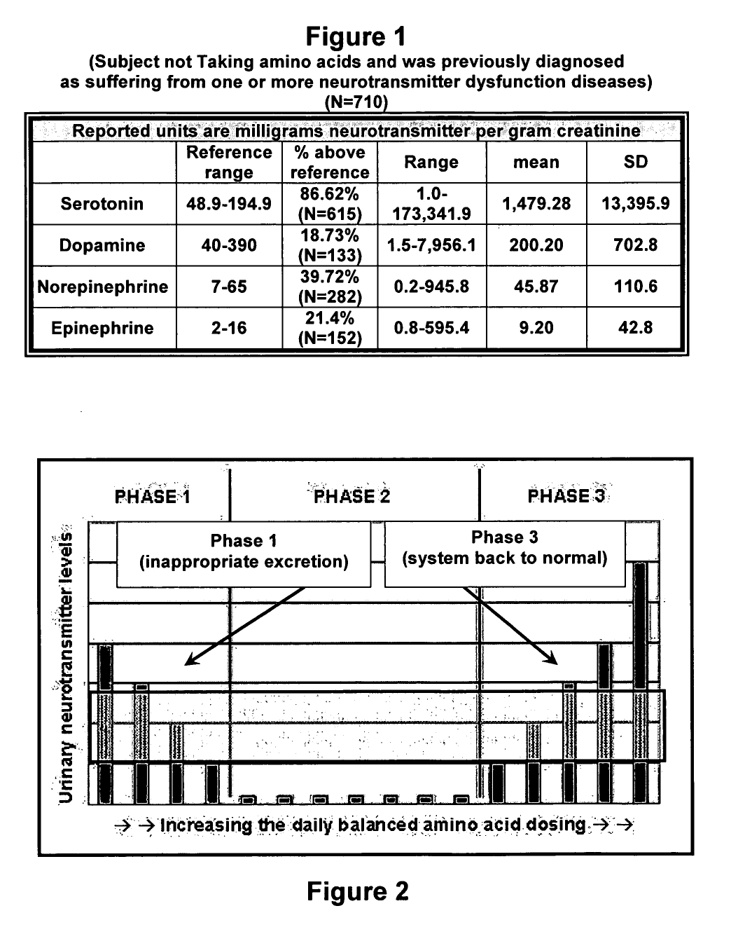

Hyperexcretion appears to be due to inappropriate excretion of neurotransmitters by the kidneys. As will be discussed more thoroughly below, inappropriate excretion of neurotransmitters into the urine by the kidneys in subjects suffering from neurotransmitter dysfunction disease appears to be one of the sources of low levels of systemic neurotransmitters. Laboratory assay of a general population of humans reveals a relatively low incidence of urinary neurotransmitter levels above the reference range reported by the laboratory. For example, it has been observed that only 7% of the general population has increased urinary serotonin levels. To the contrary, in a population of 710 subjects diagnosed with one or more neurotransmitter dysfunction diseases diagnosed by a licensed physician and not under the treatment of amino acids precursors of the serotonin and/or catecholamine systems, 94.6% of the subjects (N=672) had at least one neurotransmitter of the serotonin or catecholamine systems at a level higher than the reference range reported by the laboratory. Specifically, as summarized in FIG. 1, 86.62% of the subjects (N=615) had urinary serotonin levels that were above the reference range reported by the laboratory, 18.73% (N=133) of the subjects had high urinary dopamine levels, 39.72% of the subjects (N=282) had high urinary norepinephrine levels, and 21.4% of patients (N=152) had high epinephrine levels. As will be discussed more thoroughly below, proper use of amino acid precursors of the serotonin and/or catecholamine systems not only controls the inappropriate excretion of the system neurotransmitters into the urine but also allows for optimal individual and group outcomes in treatment by stabling The System neurotransmitters in a therapeutic range and in the phase 3 response, which is illustrated in FIG. 2. Hyperexcretion is a consideration in baseline testing of subjects prior to initiation of amino acid treatment and the impact of hyperexcretion must be considered during interpretation of these tests.

Once a subject with hyperexcretion is under treatment with amino acids (generally in a matter of a few days, 3 to 5 days for dopamine and serotonin, 2 to 6 weeks for norepinephrine after the serotonin and dopamine are in the therapeutic range and phase 3 response, and can take up to 6 to 8 months for epinephrine and norepinephrine) the hyperexcretion problem starts to resolve and a more positive correlation between the urinary neurotransmitter assay and the systemic neurotransmitters assay is seen leading to more meaningful results in establishing optimal and therapeutic ranges which are discussed further below.

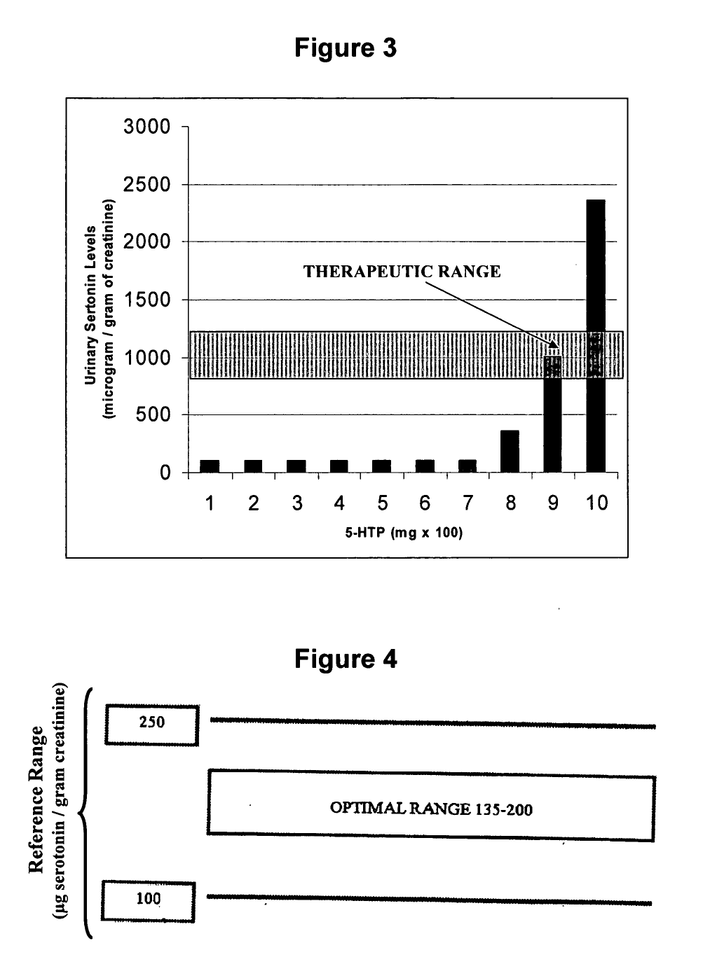

The primary application of laboratory assay of neurotransmitters of The System is to assist in establishing therapeutic levels of neurotransmitters, which correlate with the resolution of disease symptoms. Referring to FIG. 3, which illustrates the phase 2 and phase 3 response of FIG. 2, the following discussion is put forth. We use the 5-HTP component of the balanced administration of amino acids for this discussion. Similar considerations exist for L-dopa and dopamine and other amino acid precursors of The System. The horizontal axis of FIG. 3 represents the daily milligram dosing of 5-HTP in 100s of milligrams. The vertical access of FIG. 3 represents urinary serotonin levels as reported in micrograms per gram of creatinine. In a subject who is treated with a significant quantity of balanced amino acids (use of amino acid precursors of both serotonin and catecholamine systems in combination) and has laboratory assay of neurotransmitter performed frequently (with every 100 milligram increase in 5-HTP) the dose-response curve of FIG. 3 is typical, which only shows phase 2 and phase 3 of FIG. 2. The dose-response curve of FIG. 2, which includes phase 1, is also typical when a subject is treated with lower doses of amino acids and has laboratory assay of neurotransmitter performed frequently (with every 100 milligram increase in 5-HTP). As amino acid dosing is increased, as shown in FIG. 2, urinary neurotransmitter levels decrease until the phase 2 response is established. Referring to FIGS. 2 and 3, in the phase 2 response, urinary neurotransmitter levels do not change and are flat as the amino acid dosing is increased until the inflection point is arrived at which in the case of FIG. 3 is at the daily dosing of approximately 800 milligrams per day of 5-HTP, which occurs in the phase 3 response. Once the inflection point is arrived at in the phase 3 response, small increases in the amino acid precursors dosing levels lead to large increases in the urinary neurotransmitter levels. In recognizing this rapid upward inflection of the urinary neurotransmitter levels we are able to define a therapeutic range in the treatment of neurotransmitter dysfunction disease symptoms. It is noted that the amino acid dosing of precursors needs of both the catecholamine and serotonin systems vary widely in a group of subjects with regards to dosing at which the inflection point occurs. The exact urinary neurotransmitter levels of the optimal and therapeutic range may vary depending on the methodology of the laboratory doing the assay but in general the lower limits of the range need to be high enough to insure that symptoms of neurotransmitter dysfunction are under control and the top end of the therapeutic range needs to be set at such a level as to insure that the subject is not being over loaded with neurotransmitter during treatment leading to undesirable outcomes. For the purposes of the illustration in FIG. 3 the therapeutic range for serotonin is set at 800 micrograms of neurotransmitter per gram of creatinine. Other ranges exist as discussed further. The three phase response illustrated in FIG. 2 will also be discussed further below.

A further consideration of laboratory testing is illustrated in FIG. 4. The first step is to define a reference range via statistical analysis of the population as is standard practice for laboratories. In FIG. 4 the respective laboratory reference range of serotonin is defined as 100 to 250 micrograms of serotonin per gram of creatinine. It is recognized that many people with urinary neurotransmitter assay values inside of the reference range are suffering from neurotransmitter dysfunction related illness and the only way to provide effective relief of symptoms is to establish neurotransmitter levels that are higher than the reference range in what is known as the therapeutic range. The Parkinson's disease model illustrates very well why higher than normal levels are needed in many subjects not just in Parkinsonism. But still there is a subgroup of people who have no symptoms of neurotransmitter dysfunction and are functioning at a very high level. In studying this group of subjects, an optimal range was defined inside the reference range as illustrated in FIG. 4. Use of neurotransmitter assay in defining an optimal range for subjects who have no symptoms of neurotransmitter dysfunction is another application of this invention.

While the discussion so far has focused on urinary neurotransmitter assay applications the following considerations are made for salivary, serum, or other test methods that reflect the neurotransmitter status of the subject. With these other forms of assay there is also a flat dose-response curve in phase 2, as noted in FIGS. 2 and 3, to the point of inflection in phase 3 at which time small increases in amino acid dosing leads to large increases in the neurotransmitters measured. With these other methods of assay there is also an optimal range that can be defined within the reference range.

All methods of assay when used properly insure that adequate amino acid therapy is given without overloading the system with neurotransmitters. With all methods the response to a given dose of amino acids varies widely. For example, subjects have been seen who experience the inflection point of FIGS. 2 and 3 on 50 milligrams per day of 5-HTP and others who do not experience the inflection point of FIGS. 2 and 3 until 1,500 milligrams of 5-HTP per day is administered. When L-dopa and 5-HTP are used in combination for the synthesis of dopamine and serotonin respectively to affect proper balanced administration of amino acids, neurotransmitter levels of the system can be established that are well above the therapeutic range. When the other precursors of dopamine and serotonin are used, such as precursors of L-dopa and 5-HTP, in general neurotransmitter levels only into the therapeutic range can be established with a small probability of establishing neurotransmitter levels that are higher than the therapeutic range.

The invention provides the ability to optimize group results in the treatment of The System related dysfunction via a safe and effective method to gain control of The System in the treatment of dysfunction, as well as to facilitate optimal function for systems dependant on the catecholamine and/or serotonin systems for regulation and function as guided laboratory assay of neurotransmitters of The System. Laboratory values and amino acid dosing listed in this description are for obtaining optimal results in a human population. Adjustment in dosing for non-human populations should be made based on body weight and response as verified by laboratory assay.

A primary use of laboratory neurotransmitter assay is three-fold:
1. To establish a baseline assay prior to treatment with amino acids.
2. To establish a therapeutic level of neurotransmitters in treatment with amino acids whereby the neurotransmitters are high enough in the system at the low point during the day to insure that symptoms of neurotransmitter dysfunction are not present and that neurotransmitter levels are not too high in the system so as to create other problems such as serotonin syndrome and the like.
3. To establish an optimal level of neurotransmitters in those subjects not suffering symptoms of neurotransmitter dysfunction.

In order to affect the three uses of neurotransmitters described immediately above, neurotransmitter assay via serum, saliva, urine, or other methods is used, as long as considerations of the limitations of each method as previously discussed are compensated for.

For saliva assay of neurotransmitters of The System, a compensation is the need to perform several tests over a relatively short period of time (approximately 30 minutes) and averaging of the results.

For serum assay of neurotransmitters of The System, a compensation is the need to collect a sample where the subject is not disturbed so as to not affect the baseline neurotransmitter levels present just prior to collection of the sample.

For urinary assay of the neurotransmitters of The System, a compensation is a method of reporting results whereby the variability in the specific gravity of the urine is compensated for.

Methods for compensation in saliva, serum, and urine were previously discussed and the following is a discussion of interpretation and applications of neurotransmitter assay of neurotransmitters of The System.

The following laboratory value numbers are for the specific laboratory used in the research of this invention. Due to variability in assay techniques between laboratories actual values may legitimately vary from laboratory to laboratory.

“REFERENCE RANGES” are the ranges set by the individual laboratory from statistical analysis of a population of subjects based on defining the mean and standard deviation. The typical reference range is the value found in calculating two standard deviations above and below the mean. The reference range reported by each laboratory may also be unique depending on the methodology of the assay being used. An exemplary embodiment of the reference range established by a first laboratory is as follows:

Serotonin=100 to 250 micrograms of neurotransmitter per gram of creatinine.

Dopamine=100 to 250 micrograms of neurotransmitter per gram of creatinine.

Norepinephrine=25 to 75 micrograms of neurotransmitter per gram of creatinine.

Epinephrine=5 to 13 micrograms of neurotransmitter per gram of creatinine.

Another exemplary embodiment of the reference range established by a second laboratory is as follows:

Serotonin=48.9 to 194.9 micrograms of neurotransmitter per gram of creatinine.

Dopamine=40.0 to 390.0 micrograms of neurotransmitter per gram of creatinine.

Norepinephrine=7.0 to 65.0 micrograms of neurotransmitter per gram of creatinine.

Epinephrine=2.0 to 16.0 micrograms of neurotransmitter per gram of creatinine.

OPTIMAL RANGES are defined as a narrow range within the reference range where subjects with no symptoms of neurotransmitter dysfunction appear to be functioning optimally based on group observations. The optimal ranges for the neurotransmitters of The System for the first laboratory above are as follows:

Serotonin=175 to 225 micrograms of neurotransmitter per gram of creatinine.

Dopamine=125 to 175 micrograms of neurotransmitter per gram of creatinine.

Norepinephrine=30 to 55 micrograms of neurotransmitter per gram of creatinine.

Epinephrine=8 to 12 micrograms of neurotransmitter per gram of creatinine.

The optimal ranges for the neurotransmitters of The System for the second laboratory above are as follows:

Serotonin=85.6 to 175.4 micrograms of neurotransmitter per gram of creatinine.

Dopamine=50.0 to 273.0 micrograms of neurotransmitter per gram of creatinine.

Norepinephrine=8.4 to 47.7 micrograms of neurotransmitter per gram of creatinine.

Epinephrine=3.2 to 14.8 micrograms of neurotransmitter per gram of creatinine.

THERAPEUTIC RANGES are the range to be obtained in treatment to insure that resolution of symptoms is affected without overloading the system on neurotransmitters. The therapeutic ranges of the neurotransmitters of The System are as follows. It should be noted that these numbers are a relative guide only for reaching an inflection point in treatment and that the therapeutic range should not be fixed on the absolute numbers reported. Instead, the therapeutic range is specific to the respective neurotransmitter dysfunction disease. In general, the therapeutic range for serotonin in neurotransmitter dysfunction is typically 800 to 2400 micrograms of neurotransmitter per gram of creatinine and in phase 3. For example, the therapeutic range for serotonin in non-obesity neurotransmitter disease is reported at 800 to 1,200. A serotonin level of 1,600 or higher could be acceptable in some circumstances.

Serotonin=1,200 to 2,400 micrograms of neurotransmitter per gram of creatinine for treatment of obesity, obsessive compulsive disorder (COD), panic attacks and severe anxiety.

Serotonin=250 to 1,200 micrograms of neurotransmitter per gram of creatinine for disease not related to obesity. Such as in conditions that respond relatively early on in treatment such as migraine headaches and some chronic pain states.

In general, the therapeutic range for dopamine in neurotransmitter dysfunction is typically 200 to 600 micrograms of neurotransmitter per gram of creatinine.

Dopamine (in treatment of Parkinsonism) <20,000 micrograms of neurotransmitter per gram of creatinine and treatment decisions are driven by clinical outcomes.

In general, the therapeutic range for norepinephrine in neurotransmitter dysfunction is typically 7 to 65 micrograms of neurotransmitter per gram of creatinine.

In general, the therapeutic range for epinephrine in neurotransmitter dysfunction is typically 2 to 16 micrograms of neurotransmitter per gram of creatinine.

The goal of treatment is to establish neurotransmitter levels of The System in the optimal range for subjects with no symptoms of neurotransmitter dysfunction and in the therapeutic range for subjects suffering from symptoms of neurotransmitter dysfunction. To affect establishment of neurotransmitters in the desired range, adjusting the dosing of amino acids is affected as described by the Applicant in U.S. Patent Application Publication No. U.S. 2003/0181509 A1, published Sep. 25, 2003, which is incorporated by reference in its entirety herein.

In certain embodiments, the dosing ratio of the amino acid precursors L-tyrosine and 5-HTP for optimal results is generally 10:1 on a milligram-to-milligram basis and the ratio of L-dopa to 5-HTP for optimal results when used is 1:3 or within 85% of these values as discussed in U.S. Patent Application Publication No. U.S. 2003/081509 A1, published Sep. 25, 2003. The dosing ratio of phenylalanine to 5-HTP for optimal results, when used, is 10:1; and the dosing ratio of N-acetyl-L-tyrosine to 5-HTP for optimal results, when used, is 5:1 or within 85% of these values as discussed in U.S. Patent Application Publication No. U.S. 2003/081509 A1, published Sep. 25, 2003. Other amino acid precursors of dopamine and serotonin may be used with considerations for ratios to obtain optimal results, but if the goal is optimal results with group treatment dosing ratios close (within 85%) to these should generally be used. Proper use of amino acid ratios is referred to as “balanced.”

Applications of the present invention relate to any application where the serotonin and/or catecholamine neurotransmitter systems are involved. This includes treatment in and of itself in subjects suffering from central nervous system neurotransmitter diseases, which includes, but is not limited to: depression, anxiety, panic attacks, migraine headache, obesity, bulimia, anorexia, premenstrual syndrome, menopause, insomnia, hyperactivity, attention deficit disorder (ADD), impulsivity, obsessionality, inappropriate aggression, inappropriate anger, psychotic illness, obsessive compulsive disorder (OCD), fibromyalgia, addictions, sexual dysfunction, chronic fatigue syndrome, chronic pain states, adrenal fatigue/burnout, attention deficit hyperactivity disorder (ADHD), Parkinsonism, traumatic brain injury, claustrophobia, tension headaches, nocturnal myoclonus, irritable bowel syndrome/Chron's disease, states of decreased cognitive function such as dementia and Alzheimer's disease, states associated with aging such as deterioration of organ systems innervated by the serotonin and/or catecholamine systems and deterioration of cognitive functions, hormone dysfunction problems innervated by the serotonin and/or catecholamine systems; cortisol dysfunction related problems, and neurotransmitter reaction to chronic stress.

2. Description of the Preferred Embodiment of the Invention

For neurotransmitter testing of neurotransmitters of The System, urine is collected approximately 5 to 6 hours prior to bed time and just prior to any amino acid dosing the subject may or may not be taking close to that time period. Once the urine sample is obtained, laboratory assay of the neurotransmitter of The System are performed as well as a urinary creatinine assay and the results are reported in terms of micrograms of neurotransmitter per gram of creatinine.

After there has been a start or change in the dosing of amino acid precursors of The System, the following considerations exist with regards to neurotransmitter assay.
1. It takes 3 to 5 days for serotonin levels to come to equilibrium in the urine.
2. It takes 3 to 5 days for dopamine levels to come to equilibrium in the urine.
3. It takes 2 to 6 weeks for norepinephrine to come to equilibrium in the urine after the serotonin and dopamine have been established in the therapeutic range and in the phase 3 response.
4. It can take as long as 3 to 6 months for epinephrine to come to equilibrium in the urine after the serotonin and dopamine have established in the therapeutic range and in the phase 3 response.

If the subject is not under treatment with amino acid precursors of The System at the time the sample is collected, the results are used as a baseline reference point in treatment with amino acid precursors of The System.

If the subject has no symptoms of neurotransmitter dysfunction the subject is treated with amino acids precursors of The System and the amino acid dosing increased or decreased as guided by laboratory assay of neurotransmitters of The System until reported results of laboratory assay are in the optimal range.

If the subject is suffering from symptoms of neurotransmitter dysfunction, the subject is treated with amino acid precursors of The System which are in turn increased or decreased until urinary neurotransmitter levels of The System have been established in the therapeutic range.

Retesting of the subject should be done one week or more after every dose change in the amino acid precursors takes place.

Once under treatment and optimal or therapeutic ranges have been established periodic retesting should be preformed at regular intervals. It is the preferred method to perform follow up testing every six months or sooner.

If assay of neurotransmitter reveals that the urinary neurotransmitter levels are low, the correct response is to increase the amino acid precursor dosing of The System. If assay of the neurotransmitters reveals that the urinary neurotransmitter levels are high the correct response is to decrease the amino acid precursor dosing of The System.

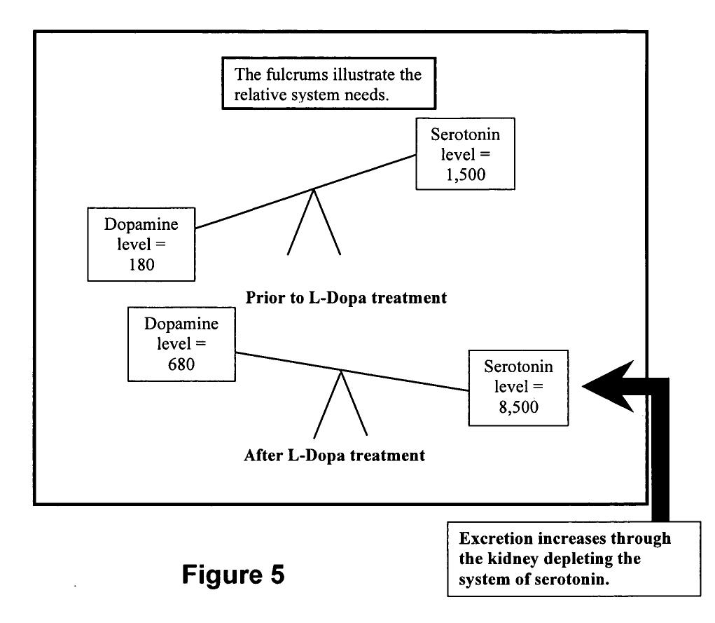

FIG. 5 illustrates the effects of using unopposed precursors of dopamine in treatment. With the addition of unopposed L-dopa the urinary excretion of serotonin increases markedly, a fact that was not previously known. The same is true with administration of unopposed precursors of the serotonin system such as 5-HTP. With the administration of 5-HTP alone there is a marked increase in the excretion of dopamine by the kidneys. These observations are of importance in establishing optimal and therapeutic ranges of neurotransmitters as guided by laboratory assay. For example, in a subject under treatment with amino acid precursors of The System who is experiencing symptoms of neurotransmitter dysfunction that are not obesity related and has a dopamine reported by assay of 150 micrograms of dopamine per gram of creatinine and a serotonin of 1,100 micrograms of serotonin per gram of creatinine and is still experiencing symptoms of neurotransmitter dysfunction, management considerations are as follows.
1. By giving more of the balanced amino acid precursor of The System the urinary levels of both serotonin and dopamine levels will rise. This may lead to resolution of symptoms as the dopamine levels are established in the therapeutic range but the serotonin levels will be higher than the therapeutic or optimal range as well.
2. By administering only amino acid precursors of dopamine the ability is gained to establish dopamine levels in the therapeutic or optimal ranges. Referring to FIG. 5, by using unopposed amino acid precursors in treatment the excretion of serotonin by the kidneys increases markedly again leading to a situation where the dopamine is in the therapeutic range and the urinary serotonin levels are higher than the therapeutic or optimal ranges.
3. The proper management of a subject with 150 micrograms of dopamine per gram of creatinine and 1,100 micrograms of serotonin per gram of creatinine level is to increase the amino acid precursors of dopamine while at the same time decreasing the amino acid precursors of serotonin to give an outcome whereby neurotransmitters of both systems are in the therapeutic or optimal ranges.

Not all neurotransmitter dysfunction related symptoms resolve in the same therapeutic ranges. In general treatment of obesity, panic disorder, and obsessive compulsive disorder require urinary serotonin levels of 1,200 to 2,400 micrograms of serotonin per gram of creatinine. For diseases other than obesity, obsessive compulsive disorder, and panic disorder, serotonin levels of 250 to 1,200 micrograms of serotonin per gram of creatinine are the usual range. In the treatment of Parkinsonism the therapeutic range for serotonin is 250 to 2,400 micrograms per gram of creatinine and the therapeutic range is to elevate the dopamine levels high enough to get the symptoms of Parkinsonism under control. In general, in treatment of Parkinsonism the therapeutic range is to keep the dopamine levels less than 20,000 micrograms of dopamine per gram of creatinine. Although preferred numbers for a therapeutic range are provided here, it should be understood that variance in lab techniques may change these numbers and that testing should be used as a guide to insure that the phase 3 inflection point has been reached without overloading the system. It should be understood that recognition of an inflection point during treatment is an important consideration.

Simply establishing urinary neurotransmitter levels in the therapeutic range may not control symptoms in all subjects. For example, in treatment of the obese subject with a urinary serotonin assay of 2,300 micrograms of serotonin per gram of creatinine and a dopamine level of 475 micrograms of dopamine per gram of creatinine with the norepinephrine and epinephrine levels in the therapeutic ranges as well and the subject is not losing weight considerations are as follows. In a case such as this from a neurotransmitter standpoint of The System, further treatment may be limited and other causes that are preventing the subject from losing weight should be considered. In most cases such as this, there is a major stressor in the subject's life that can be identified which is distracting them from doing the things they need to do to be successful at weight loss. Considerations such as this also apply to other neurotransmitter dysfunction symptoms of illness.

Referring now to the data summarized in FIGS. 1 and 6-13, the data is from a database of subjects who have been diagnosed by a licensed healthcare provider as having one or more neurotransmitter dysfunction(s) associated with the serotonin and/or catecholamine systems. The database fields include:
1. The neurotransmitter diagnosis made by a licensed healthcare provider;
2. Amino acid dosing of amino acid precursors of the serotonin and dopamine neurotransmitters;
3. Laboratory assay of urinary serotonin and catecholamine neurotransmitter levels;
4. The subject's sex;
5. The subject's age and date of birth;
6. The subject's weight;
7. The subject's height;
8. Any allergies the subject might have;
9. Any medications (prescription and/or non-prescription) with dosing that the subject might be taking;
10. Type of license held by the caregiver; and
11. The subject's state and zip code of residence.

Also referring to the summarized date in FIGS. 1 and 6-13, the neurotransmitter analysis performed was subjected to the following criteria:
1. Urinary neurotransmitter reference ranges reported by the laboratory were determined after precision and accuracy studies had validated initial calibration results;
2. The reported reference ranges of the laboratory performing the assays were within +/−2 standard deviations of the mean for the general population used in calibration;
3. The reported reference ranges are for adults (18 years of age and older);
4. Urinary neurotransmitter samples were collected at least 3 to 4 hours after a subject took a dose of amino acid precursors;
5. Urinary neurotransmitter samples were collected 5 to 6 hours before the onset of the sleep cycle (bed time);
6. Urinary neurotransmitter samples were only collected from subjects who had taken the amino acid precursors of the serotonin and dopamine neurotransmitters consistently for 7 days or more;
7. The neurotransmitters in the urinary sample were stabilized between collection and assay with 6N hydrochloric acid; and
8. Urinary dopamine, norepinephrine, and epinephrine neurotransmitter levels were assayed by Radioimmunoassay (RIA) and urinary serotonin neurotransmitter levels were assayed by ELISA.

Now referring to FIG. 1, assay results are summarized of urinary serotonin, dopamine, norepinephrine, and epinephrine neurotransmitter levels of subjects who were not taking any supplemental amino acid precursors and who also were diagnosed with one or more neurotransmitter dysfunction diseases by a licensed healthcare provider. FIG. 1 also illustrates the reference range of the urinary neurotransmitters of the serotonin and catecholamine systems as reported by the laboratory.

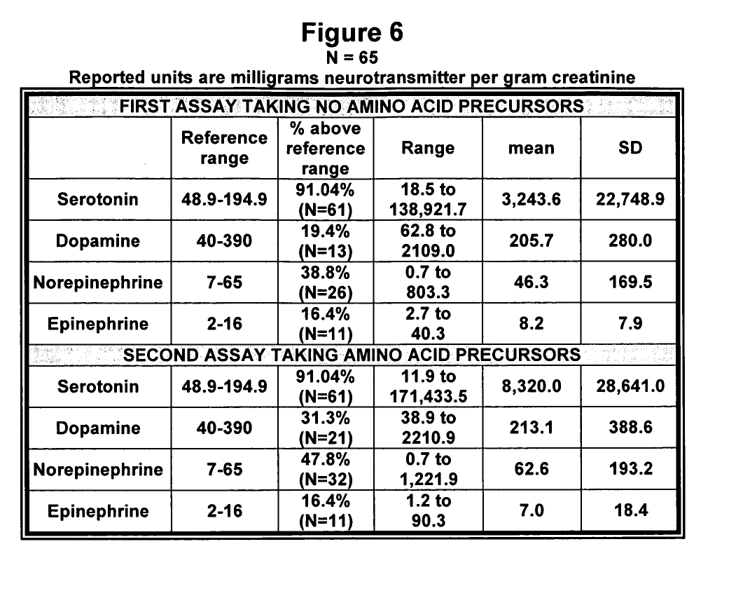

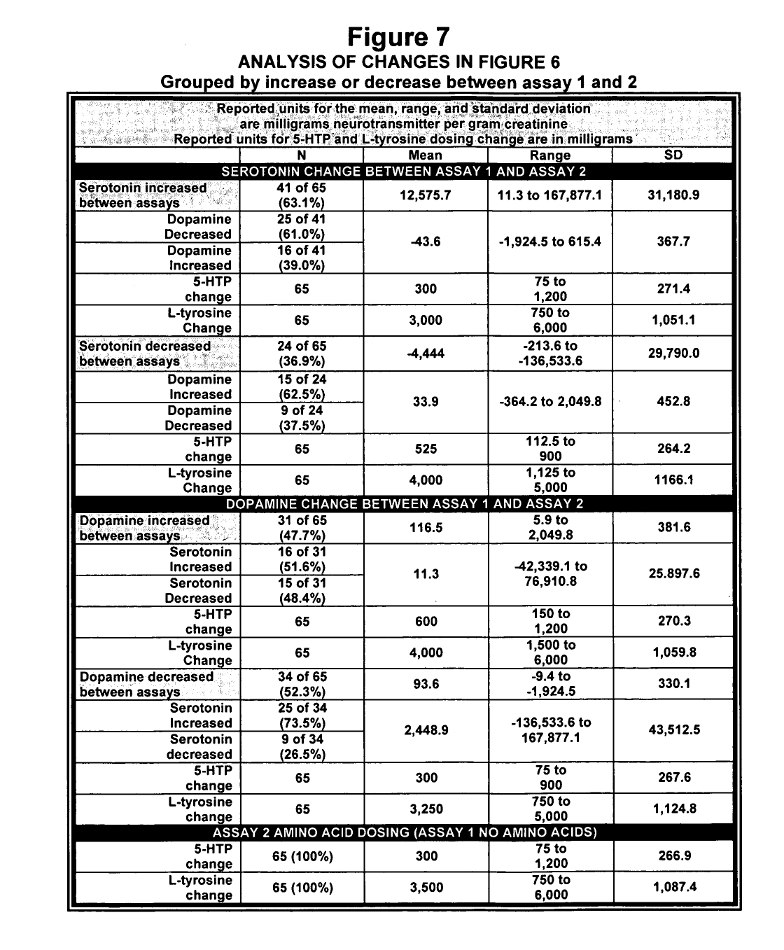

Referring now to FIGS. 3 and 4, results of two urinary neurotransmitter assays of serotonin and catecholamine levels are summarized of 65 subjects who were diagnosed with one or more neurotransmitter dysfunction diseases by a licensed healthcare provider. The first assay was performed while the subjects were not taking any supplemental amino acid precursors of serotonin and dopamine, and the second assay was performed with the subjects taking supplemental amino acid precursors of serotonin and dopamine for at least seven days. FIG. 6 illustrates the percentage of subjects who had serotonin and catecholamine neurotransmitter levels higher than the laboratory reference range when not taking any supplemental amino acid precursors of serotonin and dopamine (first assay) and when the subjects were taking supplemental amino acid precursors of serotonin and dopamine (second assay). FIG. 6 also provides statistical data of the serotonin and catecholamine neurotransmitter levels for both assays. FIG. 7 summarizes the observed changes in serotonin and dopamine neurotransmitter levels observed between the first assay and the second assay.

As illustrated in FIG. 7, the observed changes between the first and second assay can be grouped into four categories: (i) serotonin neurotransmitter levels increased between assays; (ii) serotonin neurotransmitter levels decreased between assays; (iii) dopamine neurotransmitter levels increased between assays; and (iv) dopamine neurotransmitter levels decreased between assays. In the first group, serotonin neurotransmitter levels increased between assays, an increase in urinary serotonin neurotransmitter levels is the independent variable between assay 1 and assay 2 in FIG. 6. In the second group, serotonin neurotransmitter levels decreased between assays, a decrease in urinary serotonin neurotransmitter levels is the independent variable between assay 1 and assay 2 in FIG. 6. In the first and second group, the dependent variables reported include changes in urinary dopamine neurotransmitter levels between assay 1 and assay 2, an increase in 5-HTP amino acid dosing between assay 1 and assay 2, and an increase in tyrosine dosing between assay 1 and assay 2. In the third group, dopamine neurotransmitter levels increased between assays, an increase in urinary dopamine neurotransmitter levels is the independent variable between assay 1 and assay 2 in FIG. 6. In the fourth group, dopamine neurotransmitter levels decreased between assays, a decrease in urinary dopamine neurotransmitter levels is the independent variable between assay 1 and assay 2 in FIG. 6. In the third and fourth group, serotonin the dependent variables reported include changes in urinary serotonin neurotransmitter levels between assay 1 and assay 2, an increase in 5-HTP amino acid dosing between assay 1 and assay 2, and an increase in tyrosine dosing between assay 1 and assay 2.

From the statistical analysis provided in FIG. 7, there appears to be no predictability in neurotransmitter level outcomes in changing amino acid doing in subjects taking amino acid precursor combinations of serotonin and dopamine where the dosing of the precursors is changed between assay 1 and assay 2 (N=65). The statistical analysis provided in FIG. 7 reveals the following between assay 1 of FIG. 6, where subjects were taking no amino acid precursors, and assay 2 of FIG. 6, where subjects were taking supplemental amino acid precursors of serotonin and dopamine:
1. In subjects where urinary serotonin increased between assays, 61% of the subjects had decreased dopamine neurotransmitter levels while 39% of the subjects had increased dopamine neurotransmitter levels;
2. In subjects where urinary serotonin decreased between assays, 62.5% of the subjects had increased dopamine neurotransmitter levels while 37.5% of the subjects had decreased dopamine neurotransmitter levels;
3. In subjects where urinary dopamine increased between assays, 51.6% of the subjects had increased serotonin neurotransmitter levels while 48.4% of the subjects had decreased serotonin neurotransmitter levels; and
4. In subjects where urinary dopamine decreased between assays, 73.5% of the subjects had increased serotonin neurotransmitter levels while 26.5% of the subjects had decreased serotonin neurotransmitter levels.

The statistical analysis provided in FIG. 7 and the preceding discussion illustrates that the predictability of determining an individual or group response of urinary neurotransmitter levels between a first assay, where the subject is not taking any supplemental amino acid precursors of the serotonin and dopamine neurotransmitters, and a second assay, where the subject is taking supplemental amino acid precursors of the serotonin and dopamine neurotransmitters, is very poor.

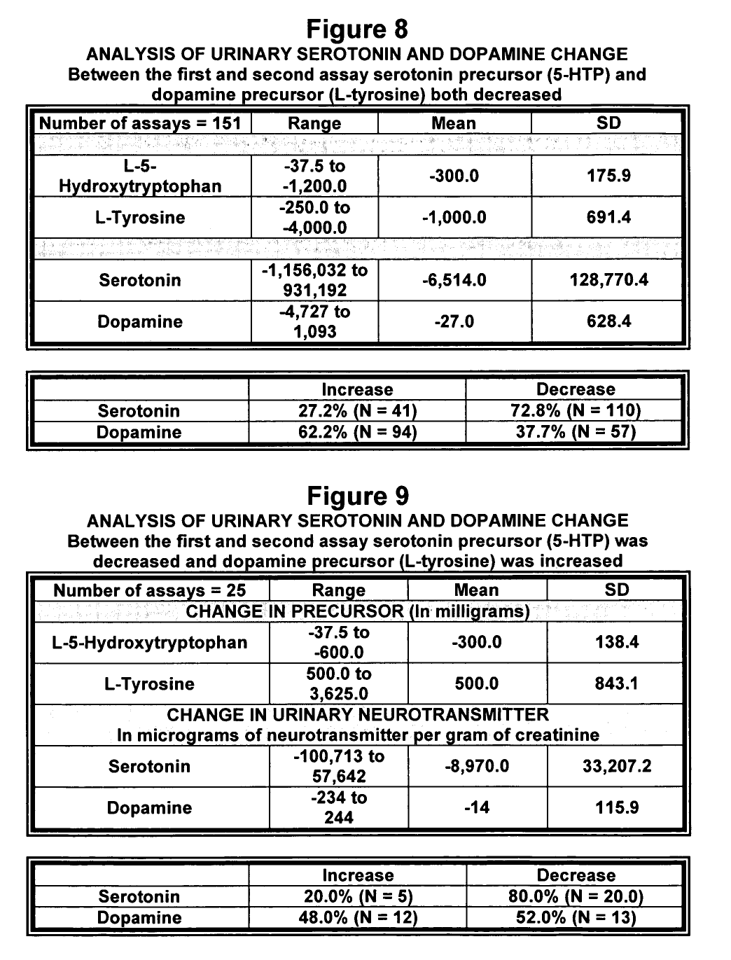

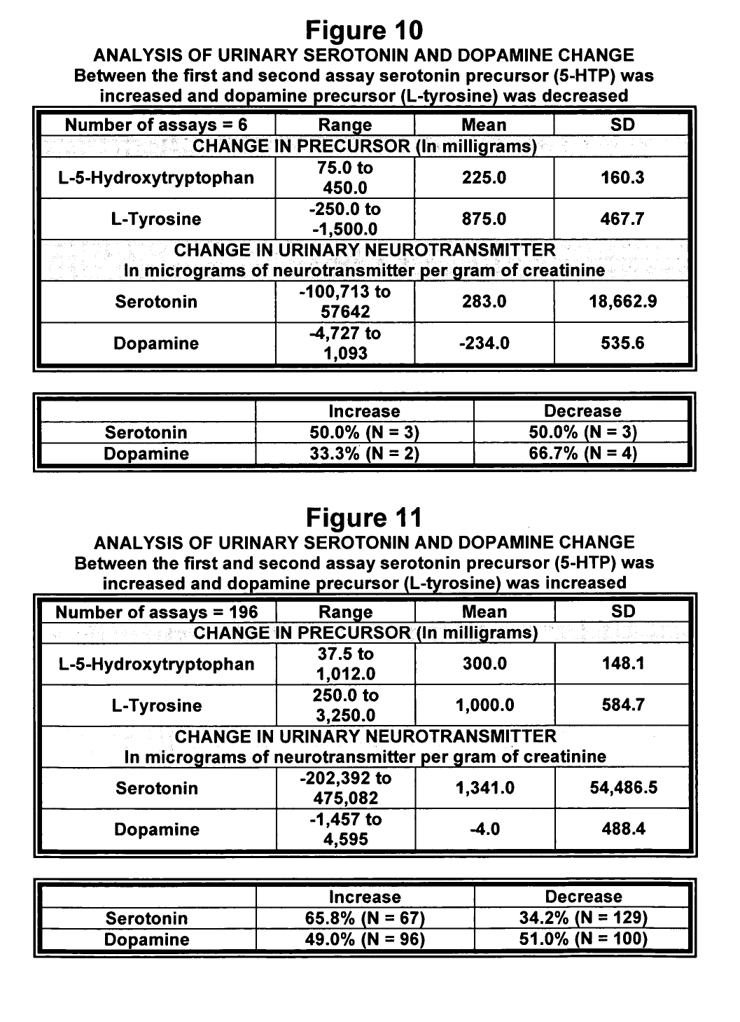

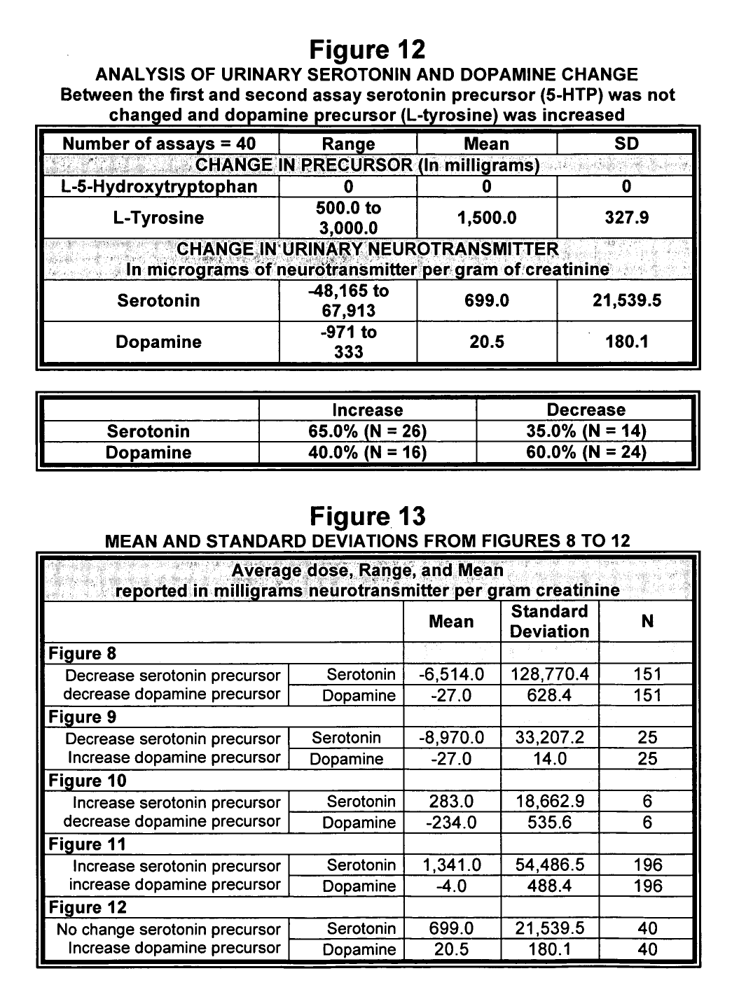

Now referring to FIGS. 8-13, results of two urinary neurotransmitter assays of serotonin and catecholamine levels are summarized of subjects who were diagnosed with one or more neurotransmitter dysfunction diseases by a licensed healthcare provider. The first assay was performed while the subjects were taking supplemental amino acid precursors of serotonin and dopamine. The second assay was performed after the subject's supplemental amino acid precursor dosing of serotonin and dopamine were changed to move the urinary serotonin and dopamine neurotransmitter levels closer to the ranges of 300 to 600 micrograms of dopamine per gram of creatinine and 800 to 2400 micrograms of serotonin per gram of creatinine and in the third phase of FIG. 2.

FIG. 8 provides a statistical analysis of 151 subjects where the dosing of both amino acid precursors (5-HTP and L-Tyrosine) of the serotonin and dopamine neurotransmitters were decreased between the first and second assay. As summarized in FIG. 8, 27.2% (N=41) of the subjects had increased serotonin neurotransmitter levels while 78.8% (N=110) of the subjects had decreased serotonin neurotransmitter levels; and 62.2% (N=94) of the subjects had increased dopamine neurotransmitter levels while 37.7% (N=57) of the subjects had decreased dopamine neurotransmitter levels.

FIG. 9 provides a statistical analysis of 25 subjects where the dosing of amino acid precursor (5-HTP) of the serotonin neurotransmitter was decreased and the dosing of the amino acid precursor (L-Tyrosine) of the dopamine neurotransmitter was increased between assays. As summarized in FIG. 9, 20.0% (N=5) of the subjects had increased serotonin neurotransmitter levels while 80.0% (N=20) of the subjects had decreased serotonin neurotransmitter levels; and 48.0% (N=12) of the subjects had increased dopamine neurotransmitter levels while 52.0% (N=13) of the subjects had decreased dopamine neurotransmitter levels.

FIG. 10 provides a statistical analysis of 6 subjects where the dosing of amino acid precursor (H-HTP) of the serotonin neurotransmitter was increased and the dosing of the amino acid precursor (L-Tyrosine) of the dopamine neurotransmitter was increased between assays. As summarized in FIG. 10, 50.0% (N=3) of the subjects had increased serotonin neurotransmitter levels while 50.0% (N=3) of the subjects had decreased serotonin neurotransmitter levels; and 33.3% (N=2) of the subjects had increased dopamine neurotransmitter levels while 66.7% (N=4) of the subjects had decreased dopamine neurotransmitter levels.

FIG. 11 provides a statistical analysis of 196 subjects where the dosing of both amino acid precursors (5-HTP and L-Tyrosine) of the serotonin and dopamine neurotransmitters were increased between assays. As summarized in FIG. 11, 65.8% (N=67) of the subjects had increased serotonin neurotransmitter levels while 34.2% (N=129) of the subjects had decreased serotonin neurotransmitter levels; and 49.0% (N=96) of the subjects had increased dopamine neurotransmitter levels while 51.0% (N=100) of the subjects had decreased dopamine neurotransmitter levels.

FIG. 12 provides a statistical analysis of 40 subjects where the dosing of the amino acid precursor (5-HTP) of the serotonin neurotransmitter was not changed and the dosing of the amino acid precursor (L-Tyrosine) of the dopamine neurotransmitter was increased between assays. As summarized in FIG. 12, 65.0% (N=26) of the subjects had increased serotonin neurotransmitter levels while 35.0% (N=14) of the subjects had decreased serotonin neurotransmitter levels; and 40.0% (N=16) of the subjects had increased dopamine neurotransmitter levels while 60.0% (N=24) of the subjects had decreased dopamine neurotransmitter levels.

Referring to FIG. 13, the statistical analysis of FIGS. 8-12 are summarized. FIGS. 8-13 and the preceding discussion illustrate that the predictability of determining an individual or group response of urinary neurotransmitter levels between two assays where the subject is taking a dosing of supplemental amino acid precursors of the serotonin and dopamine neurotransmitters and the dosing is changed between the two assays is very poor.

However, according to certain embodiments, the predictability of determining an individual or group response of urinary serotonin and/or dopamine neurotransmitter levels between two assays is possible when it is properly determined that the subject is in one of three phases of urinary neurotransmitter response to amino acid precursor administration, as illustrated in FIG. 2. Now referring to FIG. 2, it is illustrated that there are at least three phases (referred to as phase 1, phase 2, and phase 3) of urinary neurotransmitter response to amino acid precursor administration. In phase 1, the urinary neurotransmitter level drops as the amino acid precursor dosing is increased. In phase 2, the urinary neurotransmitter level remains relatively constant as the urinary amino acid dosing is increased. In order for urinary neurotransmitters to be in phase 2, subtherapeutic urinary neurotransmitter levels must exist. In phase 3, the urinary neurotransmitter level increases as the amino acid precursor dosing is increased.

In order to obtain optimal results, typically the urinary serotonin and dopamine levels must be in the therapeutic range and in the third phase. Once the urinary serotonin and dopamine levels are in the therapeutic range and in the third phase, it may take 2 to 6 weeks for the norepinephrine to equilibrate and 3 to 6 months for the epinephrine to equilibrate (although in a limited number of cases it has been observed that it can take as long as 8 to 9 months for the epinephrine to equilibrate). This phenomenon has been observed in other life forms where the serotonin and catecholamine systems with renal systems exist (i.e., cats, dogs, and horses).

As previously discussed, the amino acid precursors of the serotonin system must be in balance with the amino acid precursors of the catecholamine system to optimally treat neurotransmitter dysfunction. While previous teachings indicated that a ratio of the amino acid precursor of the serotonin system and the catecholamine system were needed for optimal results, which was typically a 10 to 1 ratio of the dopamine to serotonin amino acid precursor, in some embodiments the proper use of laboratory testing allows for individual refinement of the ratio between the serotonin and catecholamine precursors allowing for improved outcomes.

In order to achieve the necessary refinement of the ratio between the serotonin and catecholamine precursors for the treatment of neurotransmitter dysfunction, an understanding of the basic laboratory testing results that are observed with measurement of urinary master neurotransmitters is necessary.

In certain embodiments, the phase of the urinary neurotransmitters in response to amino acid therapy can properly be determined by obtaining two laboratory assays on two separate days with the subject taking a different amino acid dosing of serotonin and catecholamine precursors for each test. In general, at least 7 days must elapse between amino acid dosing changes and laboratory assay, but shorter time periods may be indicated at the decision of a caregiver. The laboratory assays should also be performed on samples that are collected at least 3 to 5 days after the last change in amino acid precursor dosing or start in amino acid precursor dosing of the serotonin and dopamine neurotransmitters. If during the first assay the subject was taking supplemental amino acid precursors of serotonin and dopamine neurotransmitters, then it is preferable that the second assay is performed after the dosing of the supplemental amino acid precursors has changed. The change in supplemental amino acid precursor may be for the serotonin neurotransmitter and/or the dopamine neurotransmitter. If both of the supplemental amino acid precursors of the serotonin and dopamine neurotransmitters are changed, it is preferable that the dosing of both supplemental amino acid precursors is increased or the dosing of both supplemental amino acid precursors is decreased. The subject also should have not missed any dosing of amino acid precursors in the 3 to 5 day period before the sample is collected.

In order to obtain urinary serotonin and dopamine levels in the therapeutic range, which relieves a significant amount of symptoms in patients, optimal results are only typically obtained when the urinary serotonin and dopamine are in “the therapeutic range and both in the phase 3 response.” Once the urinary serotonin and dopamine are in the therapeutic range and in the phase 3 response, conditions are arrived at that correlate highly with resolution of disease symptoms, optimal feeling of wellness and well being, optimal individual outcomes, and optimal group outcomes.

A relief of neurotransmitter dysfunction symptoms may also be obtained during the process of moving the urinary serotonin and dopamine neurotransmitter levels in balance toward the therapeutic range and phase three response without actually reaching the therapeutic range and phase three response. For example, it has been observed that a subject suffering from migraines has obtained relief of symptoms once the urinary neurotransmitter levels of serotonin and dopamine were less than 1000 and 500 micrograms of neurotransmitter per gram of creatinine, respectively, even though the subject was still in the phase 1 response and had inappropriate urinary excretion of the serotonin and dopamine neurotransmitters. While the relief of symptoms associated with neurotransmitter dysfunction may be observed in any of the three phases, the administration of amino acid precursors of serotonin and dopamine in balance as guided by laboratory assay is a very useful technique and therapy to establish urinary serotonin and dopamine levels that are in the therapeutic range and in the phase 3 response, which are the key to optimal outcomes.

In certain embodiments, as the amino acid dosing of balanced amino acid precursors are changed, the urinary serotonin and dopamine neurotransmitter level results discussed below may be observed. These laboratory assay results will guide the proper treatment needed in order to alleviate the inappropriate excretion of urinary serotonin and catecholamine neurotransmitters. These laboratory assay results will also guide the proper treatment needed to get both of the urinary serotonin and dopamine neurotransmitter levels into the therapeutic range and in the phase 3 response.

In subjects diagnosed as suffering from one or more neurotransmitter dysfunction diseases and who have had a first laboratory assay performed after taking a first dosing of amino acid precursors of serotonin and dopamine neurotransmitters and then have had a second laboratory assay performed after the amino acid precursor dosing has changed, three distinct phase responses exist, which are illustrated in FIG. 2, and are explained as follows:

1. Phase 1:
a. Where an increase in amino acid precursor dosing of either the serotonin amino acid precursor and/or the dopamine amino acid precursor causes a decrease in the second assay urinary serotonin or dopamine neurotransmitter levels.
b. Where a decrease in amino acid dosing of either the serotonin amino acid precursor and/or the dopamine amino acid precursor causes an increase in the second assay urinary serotonin or dopamine neurotransmitter levels.

2. Phase 2:
a. Where the first assay urinary serotonin and/or dopamine neurotransmitter levels are less than the therapeutic range and changes in the dosing of the amino acid precursors of serotonin and/or dopamine cause no changes above the lower end of the therapeutic range in the second assay urinary serotonin or dopamine neurotransmitter levels.

3. Phase 3:
a. Where an increase in amino acid precursor dosing of either the serotonin amino acid precursor and/or the dopamine amino acid precursor causes an increase in the second assay urinary serotonin or dopamine neurotransmitter levels.
b. Where a decrease in the amino acid precursor dosing of either the serotonin amino acid precursor and/or the dopamine amino acid precursor causes a decrease in the second assay urinary serotonin or dopamine neurotransmitter levels.

If the dosing of amino acid precursors of serotonin and dopamine are increased and the urinary neurotransmitter levels of serotonin and dopamine both drop, both the urinary serotonin and dopamine levels are in phase 1 response. The preferred treatment is to increase the amino acid precursors of both serotonin and dopamine in proper balance.

If the amino acid dosing of serotonin and dopamine precursors are increased and the urinary neurotransmitter levels of serotonin and dopamine both remain relatively unchanged (subtherapeutic), then both the serotonin and dopamine are in phase 2 response. The preferred treatment is to increase the amino acid precursors of both serotonin and dopamine in proper balance.

If the amino acid dosing of serotonin and dopamine precursors are increased and the urinary neurotransmitter levels of serotonin and dopamine both increase (subtherapeutic), then both the serotonin and dopamine are in phase 3 response. The preferred treatment is to adjust the amino acid dosing upward or downward in order to stabilize the serotonin and dopamine in the phase 3 therapeutic range.

If the amino acid dosing of serotonin and dopamine precursors are increased and the urinary neurotransmitter levels of serotonin increases and dopamine decreases (subtherapeutic), then the serotonin is in phase 3 and dopamine is in phase 1. The preferred treatment is to decrease the serotonin precursor (in most cases even if it is in the therapeutic range) and increase the dopamine precursor. As discussed above, increasing only one precursor of the serotonin or dopamine systems causes both the serotonin and dopamine to increase in the urine; therefore, as the dopamine precursor is increased in a subject with a urinary serotonin in or above the therapeutic range, the serotonin precursor must be decreased to compensate and insure that urinary serotonin levels do not climb higher if that is the desire.

If the amino acid dosing of serotonin and dopamine precursors are increased and the urinary neurotransmitter levels of serotonin decreases and dopamine increases (subtherapeutic), then the serotonin is in phase 1 and dopamine is in phase 3. The preferred treatment is to decrease the dopamine precursor (in most cases even if it is in the therapeutic range) and increase the serotonin precursor. As discussed above, increasing only one precursor of the serotonin or dopamine systems causes both the serotonin and dopamine to increase in the urine; therefore, as the serotonin precursor is increased in a subject with a urinary dopamine in or above the therapeutic range, the dopamine precursor must be decreased to compensate and insure that urinary dopamine levels do not climb higher if that is the desire.

If the amino acid dosing of serotonin and dopamine precursors are increased and the urinary neurotransmitter levels of serotonin remain unchanged below the therapeutic range defined by the laboratory tests and dopamine increases (subtherapeutic), then the serotonin is in phase 2 and dopamine is in phase 3. The preferred treatment is to decrease the dopamine precursor (in most cases even if it is in the therapeutic range) and increase the serotonin precursor. As discussed above, increasing only one precursor of the serotonin or dopamine systems causes both the serotonin and dopamine to increase in the urine; therefore, as the serotonin precursor is increased in a subject with a urinary dopamine in or above the therapeutic range, the dopamine precursor must be decreased to compensate and insure that urinary dopamine levels do not climb higher if that is the desire.

If the amino acid dosing of serotonin and dopamine precursors are increased and the urinary neurotransmitter levels of serotonin increases and dopamine remain unchanged below the therapeutic range defined by the laboratory tests, then the serotonin is in phase 3 and dopamine is in phase 2. The preferred treatment is to decrease the serotonin precursor (in most cases even if it is in the therapeutic range) and increase the dopamine precursor. As discussed above, increasing only one precursor of the serotonin or dopamine systems causes both the serotonin and dopamine to increase in the urine; therefore, as the dopamine precursor is increased in a subject with a urinary serotonin in or above the therapeutic range, the serotonin precursor must be decreased to compensate and insure that urinary serotonin levels do not climb higher if that is the desire.

If the urinary serotonin or dopamine in response to amino acid precursors is assayed and determined to be in phase 3 response with one neurotransmitter in the therapeutic range and the other neurotransmitter above the therapeutic range, then the neurotransmitter precursor of the neurotransmitter in the therapeutic range must be increased as the amino acid precursor of the neurotransmitter above the therapeutic range is decreased.

In certain embodiments, both the amino acid dosing of serotonin and dopamine precursors are decreased in the second dosing to determine the phase of the serotonin and dopamine. In an alternative embodiment, only one of the amino acid precursors of serotonin or dopamine are increased to determine the phase of the serotonin and dopamine. In still another alternative embodiment, only one of the amino acid precursors of serotonin or dopamine are decreased to determine the phase of the serotonin and dopamine.

In certain embodiments, the amino acid dosing range needed to move urinary serotonin or dopamine from phase 1 to phase 3 also varies widely. For example, one observed subject who was in phase 1 response only required an amino acid dosing increase of the serotonin precursor by 150 mg to move from phase 1 to phase 3, with similar results observed for dopamine and its precursors. In contrast, other observed subjects required an amino acid dosing increase of the serotonin precursor by 2,000 mg to move from phase 1 to phase 3, with similar results observed for dopamine and its precursors. For example, some observed subjects moved between phase 1 and phase 3 with very small amino acid dosing changes (37.5 mg of 5-HTP with 375 mg of L-Tyrosine or 60 mg or L-Dopa) while other subjects required much larger amino acid dosing changes (900 mg of 5-HTP with 5,000 mg of L-Tyrosine or 900 mg of L-Dopa).

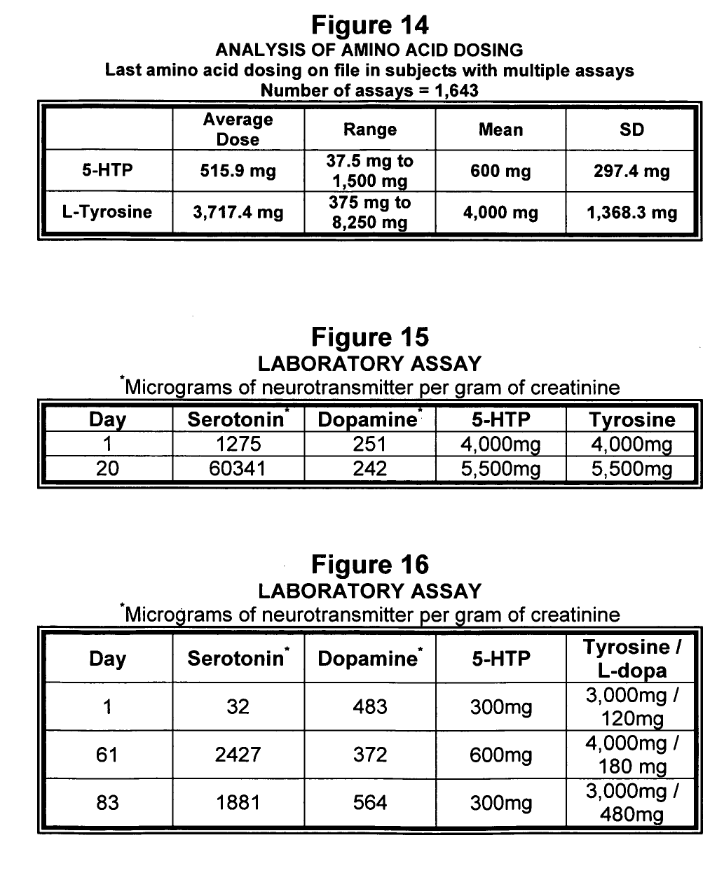

In certain embodiments, the last dosing of amino acid precursors of the serotonin and catecholamine systems also varies widely. FIG. 14 illustrates the amino acid dosing in subjects on the last neurotransmitter assay performed. The goal of adjusting the amino acid precursor dosing between assays was to obtain an outcome where the urinary dopamine was in the phase 3 response with a level of 300 to 600 micrograms of dopamine per gram of creatinine, and the urinary serotonin was also in the phase 3 response with a level of 800 to 2400 micrograms of serotonin per gram of creatinine. Other ranges within that range are also contemplated. FIG. 14 illustrates that the range for the last dosing of the 5-HTP amino acid precursor of the serotonin neurotransmitter has been observed to be between 37.5 mg and 1500 mg while the last dosing of the L-Tyrosine amino acid precursor of the dopamine neurotransmitter has been observed to be between 375 mg and 8250 mg. In certain embodiments, the preferred daily amino acid dosing in the therapeutic range and in the phase 3 response is 300 mg to 900 mg of 5-HTP and 5000 mg to 8000 mg of L-Tyrosine. Other ranges within each of those ranges are also contemplated.

In certain embodiments, the dosing needs of the amino acid precursor of serotonin are independent of the dosing needs of the amino acid precursor of dopamine in adjusting the amino acid precursors to obtain a phase 3 response for both the serotonin and dopamine neurotransmitters. By increasing the precursor of only one system (either serotonin precursor or catecholamine precursor) the other system may also be affected and show changes that are found on laboratory assay.

3. Examples

### Example 1

As illustrated in FIG. 15, a subject administered an increase in the dopamine precursor tyrosine caused a marked increase in urinary serotonin levels on the second laboratory assay without much change in the urinary dopamine levels. In this case, the difference between the first and second laboratory assays reveals that the urinary serotonin levels were in phase 3 and the urinary dopamine levels were in phase 2. As previously discussed above, the phase of urinary serotonin and dopamine levels can be ascertained by administering only one precursor of serotonin or dopamine in a patient under treatment with balanced amino acids.

### Example 2

As illustrated in FIG. 16, a subject administered an increase in the dopamine precursor L-dopa between the first and third assay increased the urinary serotonin level. This series of three tests also demonstrates a very narrow dosing range between phase 1 and phase 3 for urinary dopamine levels. In analyzing all 3 laboratory assays, the first test revealed urinary dopamine levels in phase 1, the second test revealed the urinary dopamine levels to be unknown, and the third test revealed urinary dopamine levels in phase 3. Similar results have been observed with urinary serotonin levels, which can also have a very narrow range between phase 1 and phase 3. As previously discussed, the phase of urinary dopamine or serotonin levels can be established by use of either serotonin and/or dopamine precursors in conjunction with laboratory assay.

### Example 3

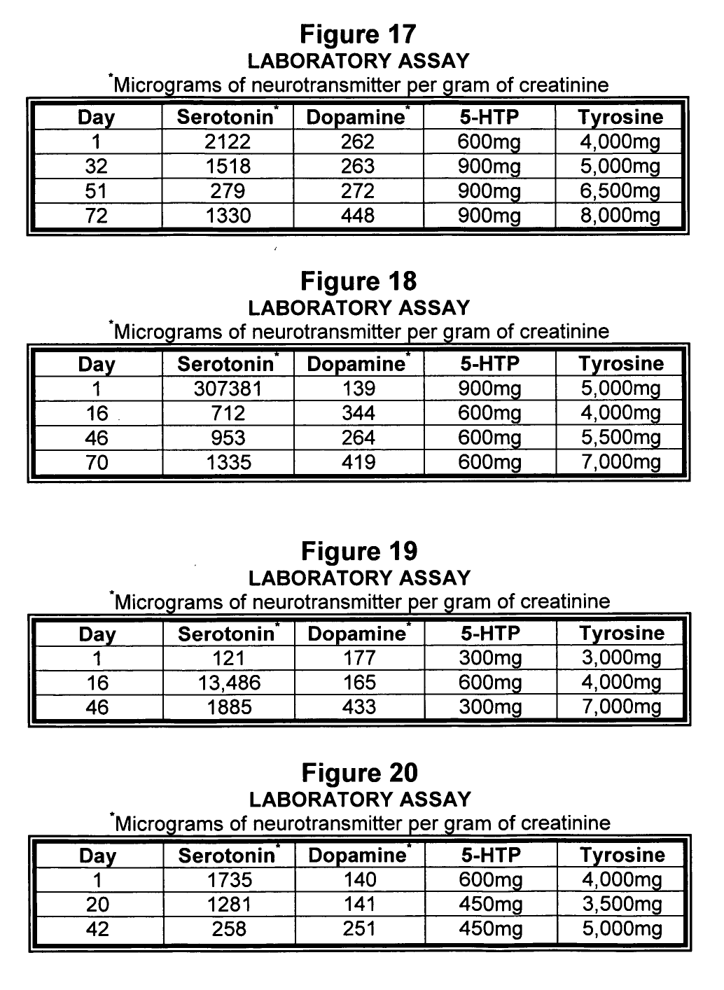

As illustrated in FIG. 17, the first and second laboratory assays reveal a urinary serotonin level in phase 1. The third laboratory assay reveals urinary serotonin levels in phase 2, and the fourth laboratory assay reveals a phase 3 response. The urinary dopamine levels are in phase 2 on the first three tests and in phase 3 on the third test. It is the teaching of this invention, which was previously discussed above, that the urinary serotonin and dopamine levels may be in different phases as serotonin and dopamine are independent of each other.

### Example 4

FIG. 18 demonstrates how adding precursors of one system can decrease the precursor needs of the other system for establishing urinary neurotransmitter level in the therapeutic range and the phase 3 response. In examining the administered laboratory assay results, test 1 reveals a phase 3 response for the urinary serotonin levels, test 2 reveals a therapeutic urinary serotonin level with the phase unknown, and test 3 reveals a phase 3 urinary serotonin level that is high (above the therapeutic range). The first 3 laboratory assays results reveals that the phase 3 therapeutic range for urinary serotonin levels is between 300 mg and 900 mg per day of 5-HTP. As the dopamine precursor tyrosine is increased, the final laboratory assay results reveal a phase 3 therapeutic urinary serotonin level with a 5-HTP dosing of 150 mg per day. As demonstrated from these laboratory assay results, the serotonin precursor needs of 5-HTP are ½ to ¼ of that which were needed with tyrosine dosing at lower levels.

### Example 5

FIG. 19 demonstrates a urinary serotonin level that is in phase 3 on the first laboratory assay result and then in a subtherapeutic range on the second test. Laboratory assays tests 3 and 4 reveals the urinary serotonin levels in phase 3. The urinary dopamine levels are in phase 2 on the first test, phase 1 on the second test, phase 2 on the third test, and finally into phase 3 therapeutic range on the last test.

### Example 6

FIG. 20 demonstrates results from a subject with a very narrow amino acid dosing range between phase 1 and phase 3. In the first assay the serotonin levels were in phase 3, in the second assay the serotonin levels were in phase 1, and in the third assay the serotonin levels were in phase 2. In this example, the change of 5-HTP dosing by 150 mg per day between test 1 and test 2 moved the urinary serotonin levels from phase 3 to phase 1.

It certain embodiments, optimal results are obtained when the dosing of amino acid precursors of serotonin and dopamine neurotransmitters are changed in such a manner that one of the precursors of serotonin or dopamine dominates the change (address primarily one system at a time even though changes will be appreciated in both systems).

The embodiments and examples described above are intended to be illustrative and not limiting. Additional embodiments are within the claims below. Although the present invention has been described with reference to specific embodiments and examples, workers skilled in the art will recognize that changes may be made in form and detail without departing from the spirit and scope of the invention. In addition, the terms including, comprising and having as used herein are intended to have broad non-limiting scope. References cited above are incorporated to the extent that they are not inconsistent with the explicit disclosure herein.

What is claimed is:

1. A method of determining a phase response of a urinary serotonin neurotransmitter and a urinary dopamine neurotransmitter in order to adjust a dosing of supplemental amino acid precursors of serotonin and dopamine neurotransmitters into a therapeutic range in a phase three response, the method comprising:
determining a first urinary serotonin and dopamine neurotransmitter levels of a subject from a first urinary assay of serotonin and dopamine neurotransmitters while the subject is taking a first dosing of supplemental amino acid precursors of serotonin and dopamine neurotransmitters;
determining a second urinary serotonin and dopamine neurotransmitter levels of a subject from a second urinary assay of serotonin and dopamine neurotransmitters while the subject is taking a second dosing of supplemental amino acid precursors of serotonin and dopamine neurotransmitters; and
determining whether the phase response of the urinary serotonin neurotransmitter and the urinary dopamine neurotransmitter is a phase one response, a phase two response, or a phase three response;
wherein the phase one response occurs where an increase in the second dosing of either the serotonin precursor and/or the dopamine precursor causes a decrease in the second urinary serotonin or dopamine neurotransmitter levels, or a decrease in the second dosing of either the serotonin precursor and/or the dopamine precursor causes an increase in the urinary serotonin or dopamine neurotransmitter levels;
wherein the phase two response occurs where an increase or decrease in the second dosing of either the serotonin precursor and/or the dopamine precursor causes the second urinary serotonin or dopamine neurotransmitter levels to remain relatively unchanged and below the therapeutic range; and
wherein the phase three response occurs where an increase in the second dosing of either the serotonin precursor and/or the dopamine precursor causes an increase in the urinary serotonin or dopamine neurotransmitter levels, or a decrease in the second dosing of either the serotonin precursor and/or the dopamine precursor causes a decrease in the urinary serotonin or dopamine neurotransmitter levels.

2. The method of claim 1, wherein the subject is a human being.

3. The method of claim 1, wherein both the serotonin and dopamine precursors of the second dosing are increased with respect to the first dosing.

4. The method of claim 1, wherein either the serotonin or the dopamine precursor in the second dosing is increased with respect to the first dosing.

5. The method of claim 1, wherein either the serotonin or the dopamine precursor in the second dosing is decreased with respect to the first dosing.

6. The method of claim 1, wherein both the serotonin and dopamine precursors in the second dosing are decreased with respect to the first dosing.

7. The method of claim 1, wherein the urinary serotonin neurotransmitter response and the urinary dopamine neurotransmitter response are both in either the phase one response, the phase two response, or the phase three response.

8. The method of claim 1, wherein the urinary serotonin neurotransmitter response and the urinary dopamine neurotransmitter response are in separate phase responses.

9. The method of claim 1, wherein the dosing of the serotonin amino acid precursor, the dopamine amino acid precursor, or both is increased based on the phase response of the urinary serotonin and dopamine.

10. The method of claim 1, wherein the dosing of the serotonin amino acid precursor, the dopamine amino acid precursor, or both is decreased based on the phase response of the urinary serotonin and dopamine.

11. The method of claim 1, wherein the serotonin amino acid precursor is tryptophan or 5-hydroxytryptophan.

12. The method of claim 11, wherein the difference between the first dosing and the second dosing is from about 37.5 mg to about 900 mg of 5-hydroxytryptophan.

13. The method of claim 11, wherein the therapeutic dosing of serotonin amino acid precursor in the phase three response is from about 37.5 mg to about 1500 mg of 5-hydroxytryptophan.

14. The method of claim 11, wherein the therapeutic dosing of serotonin amino acid precursor in the phase three response is from about 300 mg to about 900 mg of 5-hydroxytryptophan.

15. The method of claim 1, wherein the dopamine amino acid precursor is selected from the group consisting of L-tyrosine, L-dopa, N-acetyl-tyrosine, and phenylalanine.

16. The method of claim 15, wherein the difference between the first dosing and the second dosing is from about 375 mg to about 5,000 mg of tyrosine.

17. The method of claim 15, wherein the difference between the first dosing and the second dosing is from about 60 mg to about 900 mg of L-dopa.

18. The method of claim 15, wherein the therapeutic dosing of dopamine amino acid precursor in the phase three response is from about 375 mg to about 8,250 mg of tyrosine.

19. The method of claim 15, wherein the therapeutic dosing of dopamine amino acid precursor in the phase three response is from about 5,000 mg to about 8,000 mg of tyrosine.

20. The method of claim 1, wherein the a urinary dopamine neurotransmitter level of the subject in the therapeutic range in the phase three response is from about 300 to about 600 micrograms of dopamine per gram of creatinine.

21. The method of claim 1, wherein the urinary serotonin neurotransmitter level of the subject in the therapeutic range in the phase three response is from about 800 to about 2,400 micrograms of serotonin per gram of creatinine.

22. A method of adjusting a dosing of supplemental amino acid precursors of serotonin and dopamine neurotransmitters into an effective serotonin therapeutic range and an effective dopamine therapeutic range for treating a subject for neurotransmitter dysfunction, the method comprising:
comparing a first serotonin neurotransmitter level and a second serotonin neurotransmitter level as well as a first dopamine neurotransmitter level and a second dopamine neurotransmitter level to select a therapeutic dosing of supplemental amino acid precursors of serotonin and dopamine neurotransmitters wherein the first serotonin neurotransmitter level and the first dopamine neurotransmitter level are determined from a first urinary assay while the subject is taking a first dosing of supplemental amino acid precursors of serotonin and dopamine neurotransmitters and the second serotonin neurotransmitter level and the second dopamine neurotransmitter level are determined from a second urinary assay while the subject is taking a second dosing of supplemental amino acid precursors of serotonin and dopamine neurotransmitters.

23. The method of claim 22, wherein the subject is a human being.

24. The method of claim 22, wherein the serotonin amino acid precursor is tryptophan or 5-hydroxytryptophan.

25. The method of claim 22, wherein the dopamine amino acid precursor is selected from the group consisting of L-tyrosine, L-dopa, N-acetyl-tyrosine, and phenylalanine.

26. The method of adjusting a dosing of supplemental amino acid precursors of serotonin and dopamine neurotransmitters into an effective therapeutic dosing of supplemental amino acid precursors of serotonin and dopamine neurotransmitters for treating a subject for neurotransmitter dysfunction, the method comprising:
determining a urinary serotonin response and a urinary dopamine response by comparing results from a first urinary assay of serotonin and dopamine neurotransmitter levels while the subject was taking a first dosing of supplemental amino acid precursors of serotonin and dopamine neurotransmitters to the results of a second urinary assay of serotonin and dopamine neurotransmitter levels while the subject was taking a second dosing of supplemental amino acid precursors of serotonin and dopamine neurotransmitters; and
adjusting either the serotonin amino acid precursor dosing or the dopamine amino acid precursor dosing or both based on the urinary serotonin response and the urinary dopamine response.

27. The method of claim 26, wherein the subject is a human being.

28. The method of claim 26, wherein the serotonin amino acid precursor is tryptophan or 5-hydroxytryptophan.

29. The method of claim 26, wherein the dopamine amino acid precursor is selected from the group consisting of L-tyrosine, L-dopa, N-acetyl-tyrosine, and phenylalanine.
